(19) United States
(12) Patent Application Publication
(10) Pub. No.: US 2024/0091480 A1
(43) Pub. Date: Mar. 21, 2024

(54) MASK SYSTEM FOR CPAP OR BIPAP THERAPY

(71) Applicant: Jason STRADLING, Pleasant Grove, UT (US)

(72) Inventor: Jason STRADLING, Pleasant Grove, UT (US)

(21) Appl. No.: 18/468,632

(22) Filed: Sep. 15, 2023

Related U.S. Application Data
(60) Provisional application No. 63/407,126, filed on Sep. 15, 2022.

(51) Int. Cl. A61M 16/06; A61M 16/08; A61M 16/10

(52) U.S. Cl. CPC A61M 16/0616; A61M 16/0683; A61M 16/0875; A61M 16/1045

(57) ABSTRACT

The disclosure concerns mask systems for use in continuous positive airway pressure (CPAP) or bilevel positive airway pressure (BiPAP) therapy. The mask system includes: a mask comprising (i) a cup that seals around one or both of a nose and a mouth of a user, and (ii) head gear; one or more intake air hoses designed to couple with a CPAP or BiPAP machine; and one or more exhaust air hoses that are in physical contact with the one or more intake air hoses.

### RELATED APPLICATIONS

This application claims the benefit of U.S. provisional patent application 63/407,126, filed Sep. 15, 2022, titled “Mask System for CPAP or BiPAP Therapy”, the entirety of the disclosure of which is hereby incorporated herein by this reference.

### TECHNICAL FIELD

This document relates to a mask system for use during continuous positive airway pressure (CPAP) therapy or bilevel positive airway pressure (BiPAP) therapy, and more specifically relates to a mask system for CPAP or BiPAP therapy with a layered hose.

### BACKGROUND

Continuous positive airway pressure (CPAP) therapy is used to facilitate a user's breathing during sleep. A CPAP machine increases air pressure in the user's airway to prevent the airway from collapsing when the user breathes in. However, using a CPAP machine may increase discomfort for the user. In particular, many users complain that the air that is pumped into the user's mouth excessively dries out the mouth and throat. In addition, a mask is worn with the CPAP machine, causing the air breathed out by the user to remain near the user's face and body. To reduce dryness in the mouth, some CPAP machines introduce humidity in the air pumped into the user's mouth. However, doing so increases the power consumption of the CPAP machine and often causes condensation build-up within the intake hose and mask.

### SUMMARY

In some aspects, the disclosure concerns mask systems for use in continuous positive airway pressure (CPAP) or bilevel positive airway pressure (BiPAP) therapy, the mask system comprising: a mask comprising (i) a cup that seals around one or both of a nose and a mouth of a user, and (ii) head gear; an intake air hose designed to couple with a CPAP or BiPAP machine; and one or more exhaust air hoses that are in physical contact with the one or more intake air hoses.

In other aspects, the disclosure concerns methods of using a continuous positive airway pressure (CPAP) or a bilevel positive airway pressure (BiPAP) machine comprising fitting a user with a mask system and providing input air and exhaust gas from the user; wherein the mask system comprises: a mask comprising (i) a cup that seals around one or both of a nose and a mouth of a user, and (ii) head gear; one or more intake air hoses designed to couple with a CPAP or BiPAP machine; and one or more exhaust air hoses that are in physical contact with the one or more intake air hoses.

In yet other aspects, the disclosure concerns methods of treating sleep apnea comprising use of a mask system in conjunction with a continuous positive airway pressure (CPAP) or a bilevel positive airway pressure (BiPAP) machine to provide positive air pressure to a user; the mask system comprising: a mask comprising (i) a cup that seals around one or both of a nose and a mouth of a user, and (ii) head gear; one or more intake air hoses designed to couple with a CPAP or BiPAP machine; and one or more exhaust air hoses that are in physical contact with the one or more intake air hoses.

In some implementations, the one or more exhaust air hoses surround the intake air hose.

In some implementations, the cup seals around both a nose and a mouth.

In certain implementations, the head gear comprises a plurality of straps designed to loop around one or more of the user's head, neck, and ear.

The mask system may comprise a plurality of exhaust ports to pass exhaust to the one or more exhaust air hoses. The one or more exhaust air hoses are not coupled to the CPAP or BiPAP machine in some implementations.

In other implementations, the one or more exhaust air hoses are coupled to the CPAP or BiPAP machine. The exhaust air may be discarded at a position remote from the user's face and body.

In such implementations, moisture contained in the exhaust is recycled for use in humidifying air for use in the intake air hose.

The foregoing and other aspects, features, and advantages will be apparent from the descriptions, drawings, and claims.

### BRIEF DESCRIPTION OF THE DRAWINGS

Implementations will hereinafter be described in conjunction with the appended and/or included DRAWINGS, where like designations denote like elements, and:

FIG. 1 is a representation of the mask comprising a cup and headgear configured to hold the cup in the correct position on the user's head.

FIG. 2 is a further representation of the mask comprises a cup and headgear configured to hold the cup in the correct position on the user's head.

FIG. 3 is a representation of the intake hose coupled to the CPAP or BiPAP machine and the mask.

FIG. 4 is a representation of the exhaust hose(s) surrounding the intake hose.

FIG. 5 presents a further representation of a CPAP or BiPAP mask with alternate arrangements of the intake air hoses and exhaust air hoses.

FIG. 6 presents a further representation of a CPAP or BiPAP mask with alternate arrangements of the intake air hoses and exhaust air hoses.

FIG. 7 presents a further representation of a CPAP or BiPAP mask with alternate arrangements of the intake air hoses and exhaust air hoses.

### DETAILED DESCRIPTION

Detailed aspects and applications of the disclosure are described below in the following drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that implementations of the technology disclosed herein may be practiced without these specific details. It should be noted that there are many different and alternative configurations, devices and technologies to which the disclosed technologies may be applied. The full scope of the technology disclosed herein is not limited to the examples that are described below.

The singular forms “a,” “an” and “the” include plural referents unless the context clearly dictates otherwise. Thus, for example, reference to “a step” includes reference to one or more of such steps.

The word “exemplary,” “example,” or various forms thereof are used herein to mean serving as an example, instance, or illustration. Any aspect or design described herein as “exemplary” or as an “example” is not necessarily to be construed as preferred or advantageous over other aspects or designs. Furthermore, examples are provided solely for purposes of clarity and understanding and are not meant to limit or restrict the disclosed subject matter or relevant portions of this disclosure in any manner. It is to be appreciated that a myriad of additional or alternate examples of varying scope could have been presented, but have been omitted for purposes of brevity.

Throughout the description and claims of this specification, the words “comprise” and “contain” and variations of the words, for example “comprising” and “comprises”, mean “including but not limited to”, and are not intended to (and do not) exclude other components.

As required, detailed implementations of the present disclosure are included herein. It is to be understood that the disclosed implementations are merely exemplary of the invention that may be embodied in various forms. Therefore, specific structural and functional details disclosed herein are not to be interpreted as limits, but merely as a basis for teaching one skilled in the art to employ the present invention. The specific examples below will enable the disclosure to be better understood. However, they are given merely by way of guidance and do not imply any limitation.

The present disclosure may be understood more readily by reference to the following detailed description taken in connection with the accompanying figures and examples, which form a part of this disclosure. It is to be understood that this disclosure is not limited to the specific materials, devices, methods, applications, conditions, or parameters described and/or shown herein, and that the terminology used herein is for the purpose of describing particular implementations by way of example only and is not intended to be limiting of the claimed inventions. The term “plurality”, as used herein, means more than one. When a range of values is expressed, another implementation includes from the one particular value and/or to the other particular value. Similarly, when values are expressed as approximations, by use of the antecedent “about,” it will be understood that the particular value forms another implementation. All ranges are inclusive and combinable. All ranges are inclusive and combinable.

The present disclosure relates to a mask system for use with CPAP machines. As will be described in more detail below, the system is designed to reduce the negative side effects of using a CPAP machine and thus increase the comfort of the user. By making CPAP therapy more comfortable, users will be more likely to use the CPAP machine and thus live healthier lives.

Throughout the present disclosure, any reference to CPAP therapy or CPAP machines also applies to BiPAP therapy and BiPAP machines, as will be apparent to one of skill in the art. Each of the components discussed below may be implemented alone, or in conjunction with each of the other described components.

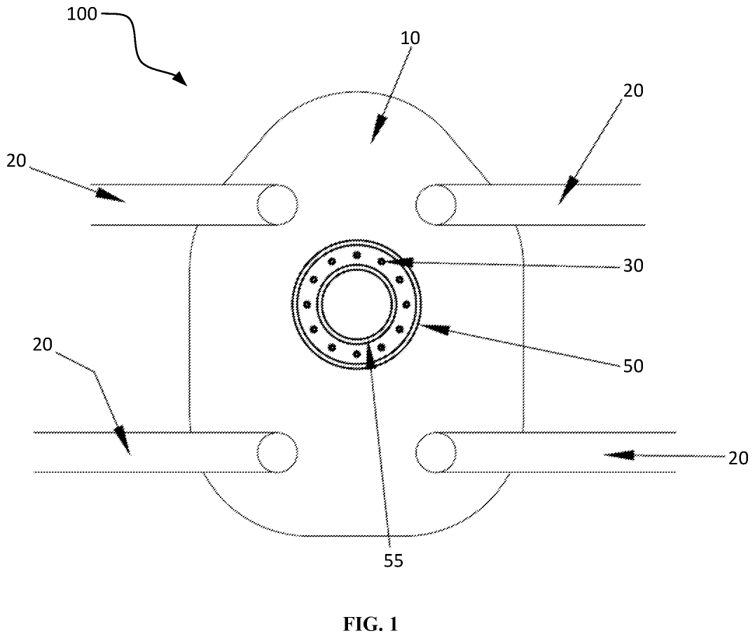

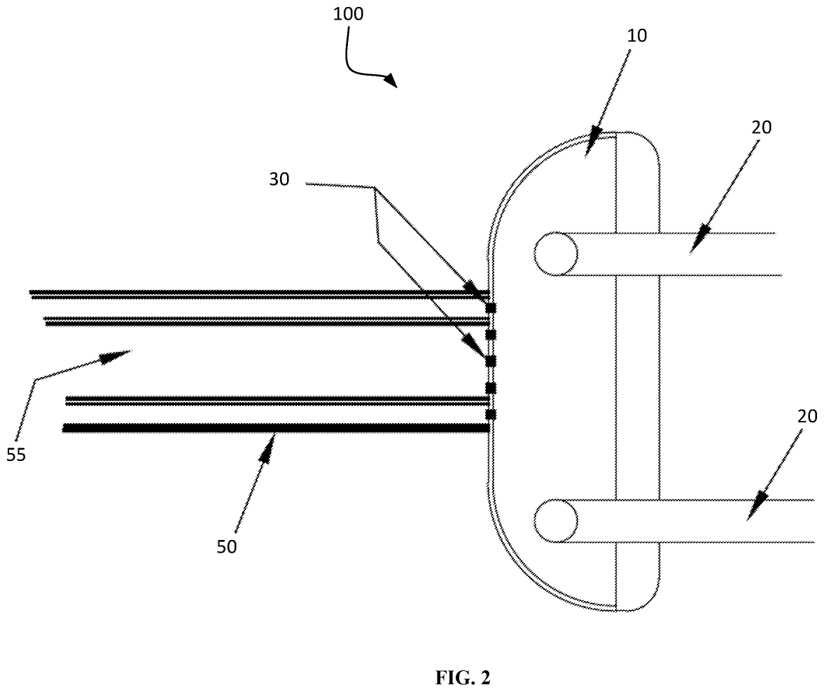

The mask system 100 disclosed herein comprises a cup 10, an intake air hose 40 and an exhaust hose 70. As shown in FIGS. 1 and 2, the mask comprises a cup 10 and headgear configured to hold the cup 10 in the correct position on the user's head.

The headgear may be a plurality of head straps or mask straps 20 configured to loop around the user's head, neck, and/or ears.

The cup 10 is configured to seal around the nose and/or mouth of the user. This allows the CPAP machine 60 to effectively push air into the airway of the user. If the cup 10 fails to properly seal around the nose and/or mouth of the user, the air pressure produced by the CPAP machine 60 is unable to open the user's airway as effectively. The cup 10 may be any shape and may be formed of any material.

The cup 10 also comprises at least one exhaust air hole 30. Some implementations of the mask have a plurality of exhaust air holes 30. The exhaust air holes 30 are configured to pass exhaled air from inside of the mask to the exterior exhaust air hose 50. The cup also comprises at least one intake air hole 40.

In some implementations, the cup 10 has a plurality of intake air holes 40. In FIGS. 1 and 2, an interior intake hose 55 is positioned within an exterior exhaust hose 50 to facilitate air intake and exhaust air output.

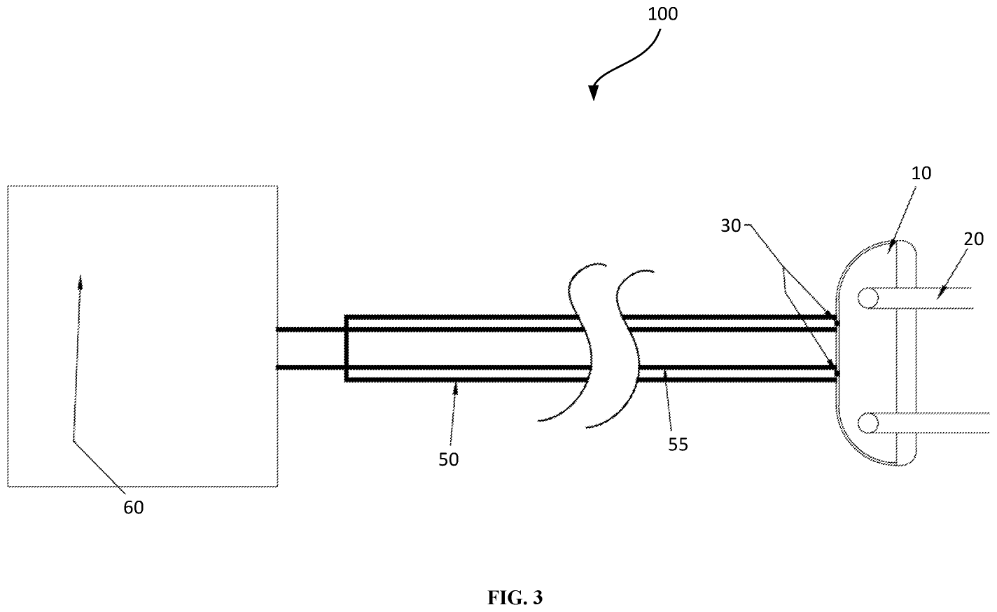

The interior intake air hose 55 is coupled to the CPAP machine 60 and to the cup 10 of the mask 100, as shown in FIG. 3, and is configured to pass positive air pressure from the CPAP machine 60 to the mask 100. Thus, the CPAP machine 60 is able to function as is known in the art in providing CPAP therapy to the user. When the air passes from the CPAP machine 60 through the interior intake air hose 55 into the mask 100, the positive air pressure created helps to keep the user's airways open for normal breathing.

The exterior exhaust air hose 50 is also coupled to the mask 100 and may or may not be coupled to the CPAP machine 60. In FIG. 3, the exterior exhaust air hose 50 ends before reaching the CPAP machine 60 and does not attach to the CPAP machine 60 allowing exhaust air to exit into the ambient air. The exterior exhaust air hose 50 is fluidly joined to the interior of the mask 100 through the plurality of exhaust air holes 30. Thus, when the user breaths out, the exhausted air is able to exit the mask 100 through the exhaust air holes 30 and the exterior exhaust air hose 50. Because the exterior exhaust air hose 50 has an end that is located away from the user of the mask 100, exhaling air through the exterior exhaust air hose 50 helps reduce the amount of exhausted air that remains near the user's face and body, and thus improves the comfort of the user both by reducing the noise from air movement near the user's face and body and by reducing the amount of air blowing on the user's face and body.

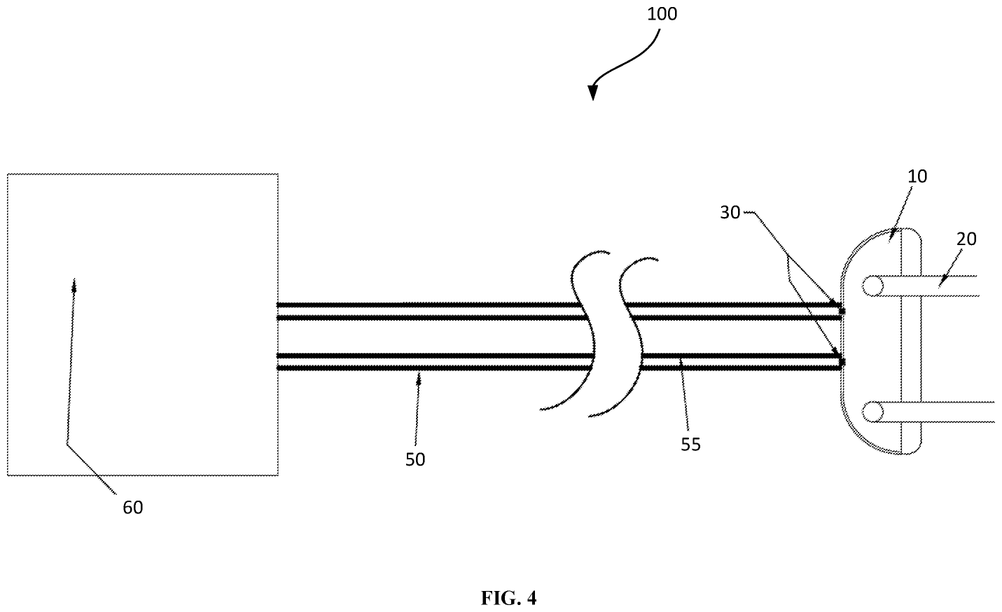

The exterior exhaust air hose 50 may surround the interior intake air hose 55. In other words, the interior intake air hose 55 extends through the exterior exhaust air hose 50 along the length of the exterior exhaust air hose 50. In some implementations, the exterior exhaust air hose 50 has a length that is equal to the length of the interior intake air hose 55, and therefore, extends from the mask 100 to the CPAP machine 60 and is coupled to the CPAP machine 60, as shown in FIG. 4.

Having the interior intake air hose 55 extend through the exterior exhaust air hose 50 further helps to improve the comfort of the user during CPAP therapy. Specifically, the exhausted air, which is typically warmer than ambient air because of the heat gained while the air was inside the user's body, helps insulate the interior intake air hose 55.

For CPAP machines 60 which introduce humidity to the air pumped into the user's mouth, insulating the interior intake air hose 55 helps to reduce condensation build-up within the interior intake air hose 55 because the intake air is maintained at a higher temperature, increasing the moisture capacity of the intake air. Additionally, having the interior intake air hose 55 extend through the exterior exhaust air hose 50 allows this reduction in condensation without increasing the power consumption of the CPAP machine 60 to the same level as is required when there is no exterior exhaust air hose 50 or when the exterior exhaust air hose 50 is separate from the interior intake air hose 55. Thus, having the interior intake air hose 55 extend through the exterior exhaust air hose 50 allows the CPAP mask system to provide moisture to the user to help limit excessive dryness from using the CPAP machine 60 without introducing some of the negative side effects of adding humidity, such as increased power consumption and condensation build-up.

In implementations of the CPAP mask system that have an exhaust hose that is coupled to the CPAP machine 60 as shown in FIG. 4, the CPAP machine 60 may include moisture recovery technology which allows the CPAP machine 60 to capture the moisture out of the exhaust air and then reuse that moisture by adding it to the intake air. This reduces the amount of water that needs to be provided to the CPAP machine 60.

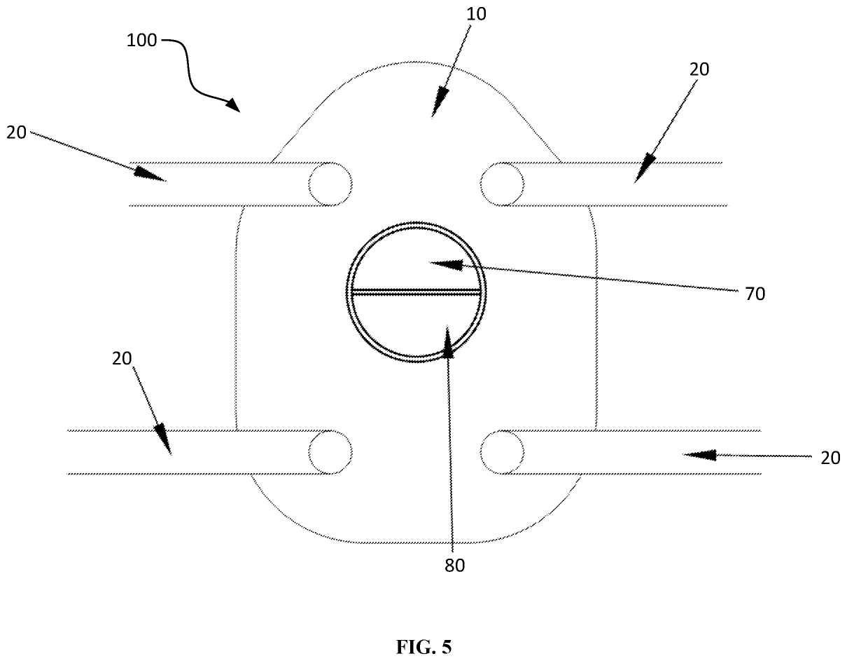

The concepts disclosed herein are not limited to the specific CPAP mask systems described above. There are other implementations of a mask system for use with CPAP machines 60. For the exemplary purposes of this disclosure, three other implementations are presented in FIGS. 5-7.

FIG. 5 illustrates a design where the exhaust air hose 70 and intake air hose 80 are a divided section of a single hose. While the figure shows one exhaust air hose 70 and one intake air hose 80 as a partitioned section of a single hose, multiple of either or both hoses may comprise a plurality of sections within the hose. In some implementations, the number of exhaust air hose 70 sections and intake air hose 80 sections may each independently be 1-20 hoses, 1-12 hoses or 1-8 hoses.

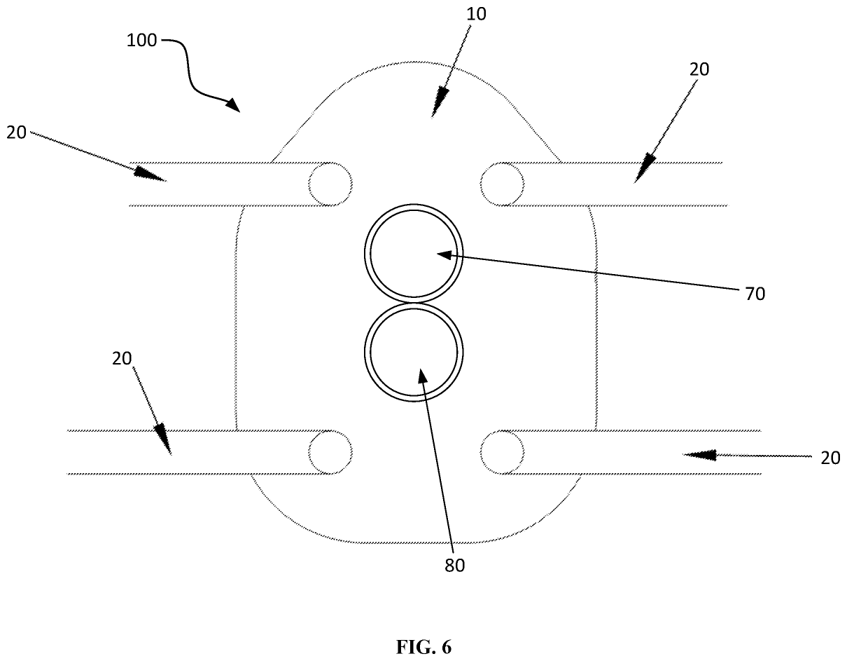

FIG. 6 illustrates a design where the exhaust air hose 70 and intake air hose 80 run parallel or adjacent to each other. While the figure shows one exhaust air hose 70 and one intake air hose 80, a plurality of each type of hose may be utilized. In some implementations, the number of exhaust air hoses 70 and intake air hoses 80 may each independently be 1-20 hoses, 1-12 hoses or 1-8 hoses.

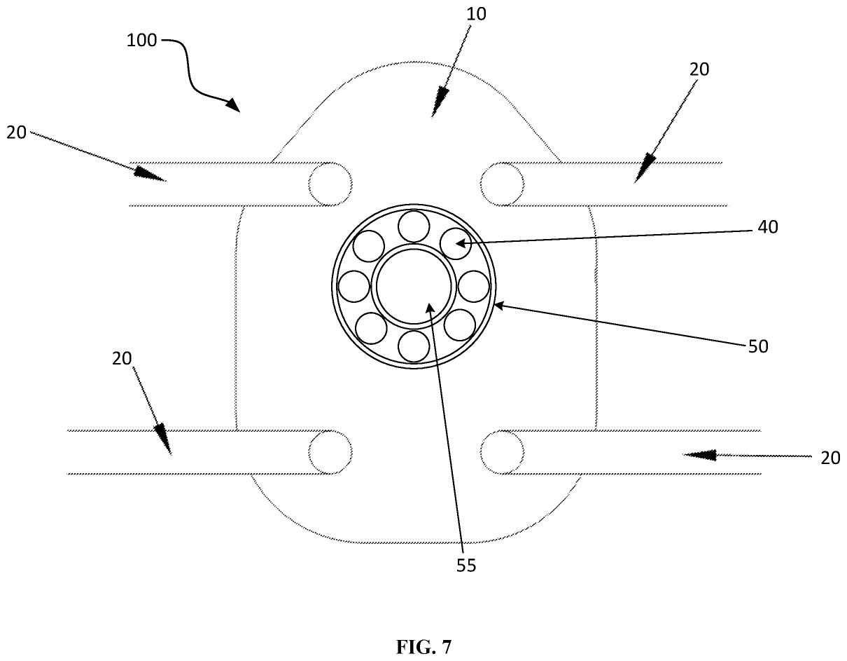

FIG. 7 illustrates a central exhaust air hose 70 that is surrounded by a plurality of intake air hoses 80. The exhaust air hose 70 and intake air hoses 80 may be a plurality of hoses or may be a single hose with multiple channels within the single hose. In some implementations, the number of intake air hoses 80 may be 2-20 hoses or 6-10 hoses.

Further implementations are within the claims.

It will also be understood that the components included in particular CPAP mask systems may be formed of any of many different types of materials or combinations that can readily be formed into shaped objects and that are consistent with the intended operation of the CPAP mask system. For example, the components may be formed of: rubbers (synthetic and/or natural) and/or other like materials; glasses (such as fiberglass), carbon-fiber, aramid-fiber, any combination therefore, and/or other like materials; elastomers and/or other like materials; polymers such as thermoplastics (such as silicone, ABS, fluoropolymers, polyacetal, polyamide, polycarbonate, polyethylene, polysulfone, and/or the like, thermosets (such as epoxy, phenolic resin, polyimide, polyurethane, and/or the like), and/or other like materials; plastics and/or other like materials; composites and/or other like materials; metals, such as zinc, magnesium, titanium, copper, iron, steel, carbon steel, alloy steel, tool steel, stainless steel, spring steel, aluminum, and/or other like materials; and/or any combination of the foregoing including, without limitation, those materials common to CPAP mask systems in the CPAP mask system industry.

Furthermore, CPAP mask system components may be manufactured separately and then assembled together, or any or all of the components may be manufactured simultaneously and integrally joined with one another. Manufacture of these components separately or simultaneously, as understood by those of ordinary skill in the art, may involve 3-D printing, extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled or removably coupled with one another in any manner, such as with adhesive, a weld, a fastener, any combination thereof, and/or the like for example, depending on, among other considerations, the particular material(s) forming the components.

In places where the description above refers to particular CPAP mask systems, it should be readily apparent that a number of modifications may be made without departing from the spirit thereof and that these implementations may be applied to other implementations disclosed or undisclosed. The presently disclosed CPAP mask systems are, therefore, to be considered in all respects as illustrative and not restrictive.

1. A mask system for use in continuous positive airway pressure (CPAP) or bilevel positive airway pressure (BiPAP) therapy, the mask system comprising:
a mask comprising (i) a cup that seals around one or both of a nose and a mouth of a user, and (ii) head gear;
one or more intake air hoses designed to couple with a CPAP or BiPAP machine; and
one or more exhaust air hoses that are in physical contact with the one or more intake air hoses.

2. The mask system of claim 1, wherein the one or more exhaust air hoses surround the intake air hose.

3. The mask system of claim 1, wherein the head gear comprises a plurality of straps designed to loop around one or more of the user's head, neck, and ear.

4. The mask system of claim 1, wherein the mask system comprises a plurality of exhaust ports to pass exhaust to the one or more exhaust air hoses.

5. The mask system of claim 4, wherein the one or more exhaust air hoses are not coupled to the CPAP or BiPAP machine.

6. The mask system of claim 4, wherein the one or more exhaust air hoses are coupled to the CPAP or BiPAP machine.

7. The mask system of claim 6, wherein moisture contained in the exhaust is recycled for use in humidifying air for use in the intake air hose.

8. A method of using a continuous positive airway pressure (CPAP) or a bilevel positive airway pressure (BiPAP) machine comprising fitting a user with a mask system and providing input air and exhaust gas from the user; wherein the mask system comprises:
a mask comprising (i) a cup that seals around one or both of a nose and a mouth of a user, and (ii) head gear;
one or more intake air hoses designed to couple with a CPAP or BiPAP machine; and
one or more exhaust air hoses that are in physical contact with the one or more intake air hoses.

9. The mask system of claim 8, wherein the one or more exhaust air hoses surround the intake air hose.

10. The method of claim 8, wherein the head gear comprises a plurality of straps designed to loop around one or more of the user's head, neck, and ear.

11. The method of claim 8, wherein the mask system comprises a plurality of exhaust ports to pass exhaust to the one or more exhaust air hoses.

12. The method of claim 11, wherein the one or more exhaust hoses are not coupled to the CPAP or BiPAP machine.

13. The method of claim 11, wherein the one or more exhaust air hoses are coupled to the CPAP or BiPAP machine.

14. The method of claim 13, wherein moisture contained in the exhaust is recycled for use in humidifying air for use in the intake air hose.

15. A method of treating sleep apnea comprising use of a mask system in conjunction with a continuous positive airway pressure (CPAP) or a bilevel positive airway pressure (BiPAP) machine to provide positive air pressure to a user; the mask system comprising:
a mask comprising (i) a cup that seals around one or both of a nose and a mouth of a user, and (ii) head gear;
one or more intake air hoses designed to couple with a CPAP or BiPAP machine; and
one or more exhaust air hoses that are in physical contact with the one or more intake air hoses.

16. The mask system of claim 15, wherein the one or more exhaust air hoses surround the intake air hose.

17. The method of claim 15, wherein the head gear comprises a plurality of straps designed to loop around one or more of the user's head, neck, and ear.

18. The method of claim 15, wherein the mask system comprises a plurality of exhaust ports to pass exhaust to the one or more exhaust air hoses.

19. The method of claim 18, wherein the one or more exhaust air hoses are not coupled to the CPAP or BiPAP machine.

20. The method of claim 17, wherein the one or more exhaust air hoses are coupled to the CPAP or BiPAP machine.
